(19) United States
(12) Patent Application Publication
(10) Pub. No.: US 2004/0116986 A1
(43) Pub. Date: Jun. 17, 2004

(54) Apparatus for lipolyusis for aesthetic treatment

(76) Inventors: Irene Cantoni, Parma (IT); Celestino Lanviti, Parma (IT)

Correspondence Address: Ladas & Parry, 26 West 61st Street, New York, NY, 10023, US

(21) Appl. No.: 10/450,642

(22) PCT Filed: Dec. 18, 2001
(86) PCT No.: PCT/IT01/00634

(30) Foreign Application Priority Data
Dec. 19, 2000 (IT) PR2000A0000067

(51) Int. Cl. A61F 2/00

(52) U.S. Cl. 607/101

(57) ABSTRACT

The inventive solution is included in the aesthetic treatment apparatus field. Subject matter of the present invention is an apparatus (1) for lipolysis for aesthetic treatment comprising at least a high frequency electrical generator (2), at least a first electrode (3), that can be applied on a zone of the body to be subjected to the aesthetic treatment and at least a second electrode (4) that can be applied on a neutral zone of the body. The apparatus (1) is also provided with means (5, 6) for the electrical connection of each electrode (3, 4) to the generator (2) and with thermo-adjustment means, operatively active at least on the first electrode (3).

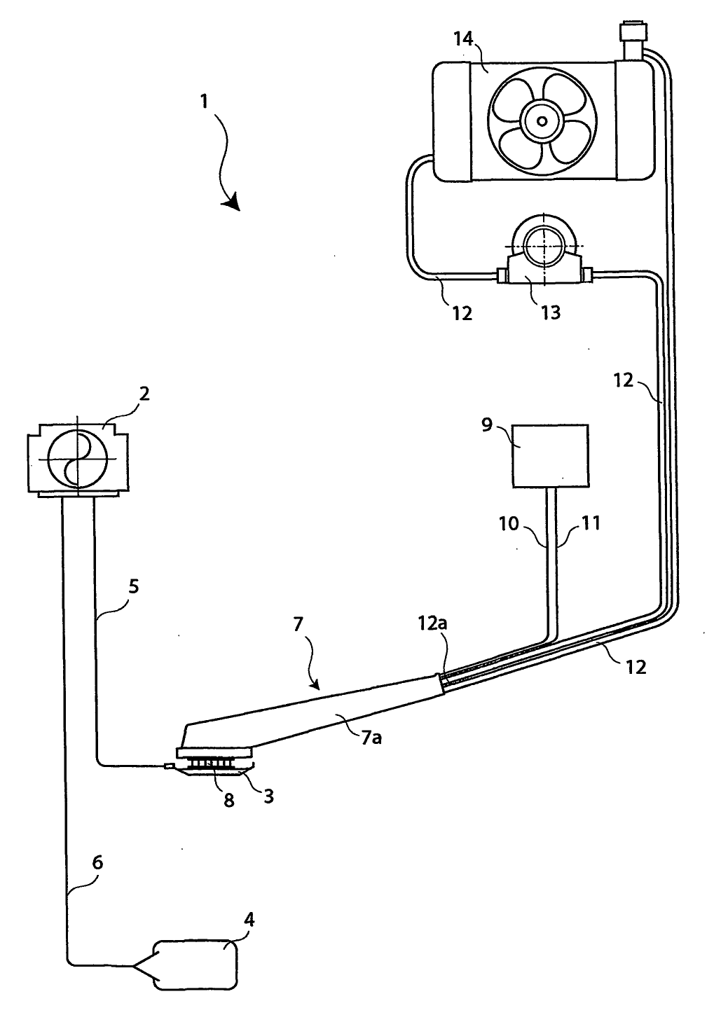

It is a specific object of the present finding an apparatus for lipolysis for aesthetic treatment.

As it is well known, aesthetic treatments allowing to reduce the subcutaneous fat include subjecting the specific body zones to a variable intensity heating.

At present, different methods and apparatuses are known for the subcutaneous fat treatment, most of them being invasive methods and providing the use of sucking tubes (e.g. lipo-suction). It is also known the use of ultrasounds, but the latter are not able to sufficiently heat the adipose tissue to dissolve the same without damaging the skin.

Object of the present invention is that of avoiding the above drawbacks, making it available an apparatus for lipolysis for aesthetic treatment able to generate caloric energy and to concentrate the same only on the in surplus adipose tissue, dissolving the same.

A further object of the present invention is that of making it available an apparatus for lipolysis for aesthetic treatment able to preserve both epidermis and muscular tissue from the heat generated to dissolve the subcutaneous fat.

Another object of the present invention is that of making it available an apparatus for lipolysis, which is easy to use, and that can be efficiently employed on all the parts of the body.

The above objects are fully reached by the apparatus for lipolysis for aesthetic treatment according to the present invention, characterised by the contents of the enclosed claims.

Further features and advantages will be more evident from the following specification of a preferred embodiment, shown, for illustrative and not limitative purposes, in the enclosed drawing, the only FIGURE of which shows a schematic view of an apparatus for lipolysis for aesthetic treatment according to the present invention.

Making reference to the FIGURE apparatus for lipolysis for aesthetic treatment according to the invention is generally indicated by reference number 1.

Apparatus 1 comprises at least a high frequency electric generator 2 and at least a first electrode 3, or “active” electrode, that can be placed on a portion of the body to be subjected to the aesthetic treatment.

Furthermore, at least a second electrode 4, or “passive” electrode” is present, to be placed on a “neutral” portion of the body (i.e. where the lipolysis effect is not necessary), close to the portion on which the first electrode 3 has been placed.

Furthermore, apparatus 1 is provided with electrical connecting means 5, 6 for connecting each electrode 3, 4 to the generator 2. Typically, said means 5, 6 are electrical cables.

The apparatus 1 according to the invention further comprises a movable containing body 7, provided with at least a handy portion or handpiece 7a. Said containing body 7 is further provided with at least a first end, in correspondence of which the first electrode 3 is provided.

Apparatus 1 further comprises thermo-adjustment means, operatively active in correspondence of the first electrode 3, i.e. the electrode destined to carry out the aesthetic treatment, said thermo-adjustment means are fixed to the containing body 7, and comprise at least a cryogen cell 8 provided in correspondence of the first end of the containing body 7 and connected with the first electrode 3, in order to maintain it at a low temperature.

A Peltier type electrical circuit, not shown in the figure, since well known in the art, is provided within the containing body 7, to make the cooling of the cryogen cell 8.

Said Peltier type electrical circuit is connected, by electrical cables 10, 11, with a supply unit 9, outside the containing body 7.

During its operation, Peltier type electrical circuit cools the first electrode 3, producing heat and, to avoid an overheating of the electrical components of the same circuit, apparatus 1 is provided with cooling means.

Particularly, said cooling means comprises at least a duct 12, defining a closed circuit containing cooling fluid. Said closed circuit provides at least a portion 12a, entering within the containing body 7 to adsorb, by cooling fluid circulating within it, the heat generated by the electric circuit.

Cooling means further comprise a pressure source 13 (e.g. a circulation pump 9 slaved to the closed circuit, in order to make the cooling fluid circulating. Further, a heat exchanger 14 is present, which is slaved to the closed circuit, and serially provided with the pressure source 13, in order to cool the cooling fluid when the same has interacted with the electric circuit.

During its use, the first electrode 3 is placed on a body portion to be subjected to the aesthetic treatment, while the second electrode 4 is placed on a contiguous neutral zone.

Thus, a preliminary cooling of the first electrode 3 (and thus of the cute) is carried out for a period of about 20 seconds, by the cryogen cell. This allows to cool the skin up to a deepness of about 2-5 mm.

Afterwards, maintaining the cryogen cell and the cooling activated, generator 2 is activated for about 20 seconds, said generator generating electromagnetic waves, preferably within the radiofrequency band, said waves concentrating between the two electrodes 3, 4, and particularly acting in a concentrated way close to the first electrode 3, since the latter has dimensions very lower than the second electrode 4, as described in the following.

This causes the heating of the adipose tissue between the epidermis and the muscular tissue, close to the first electrode.

In fact, cooling caused by the cryogen cell protects the epidermis with respect to possible bums in the electrode 3 application zone and the muscular tissue is not interested by the heating, since the latter, during the 20 seconds period, does not reach the deep muscular tissue.

At the same time of the Peltier type electrical circuit, both the pressure source 13 of the cooling circuit, starting the cooling fluid flow within the duct 12, and the heat exchanger 14, by which the cooling fluid is refrigerated, after that the same fluid interacted with the electrical circuit, are activated.

In other words, the treatment lasts 40 seconds, the first 20 seconds including only the cooling, the second 20 seconds including the “inside” heating of the zone to be treated, while the cooling goes on. In fact, the high frequency treatment does not directly heats the electrode 3, but the body portion on which it is placed.

The surface involved in the treatment, substantially is the surface of the first electrode 3, and therefore, if the zone to be treated is larger than the electrode surface (and this is the standard case), at the end of the 40 seconds period, the electrode must be displaced in another zone to be subjected to the treatment.

First electrode 3 a has a diameter of about 5 cm (and in any case a surface of about 10-30 cm²), while second electrode preferably has 25×15 cm dimensions, and in any case a surface of about 300-500 cm, i.e. more than one magnitude order bigger than the first electrode. The above causes that the radiofrequency effect is concentrated close to the first electrode and not close to the second electrode.

The heating effect penetrates at a depth of about 2-5 mm in about 20-30 seconds of application.

The shape of the electrodes is not particularly relevant and can be varied, but it is advisable that it is without sharp comers, to avoid power concentrations.

It is instead important that the dimensional ratio between the electrodes, and therefore, if a first electrode 3 having larger dimensions with respect to the above described electrode, is employed, consequently the second electrode 4 must have a corresponding bigger dimension to make it possible that an induced heating occurs only in correspondence of the first electrode.

The inventive solution allows to obtain remarkable advantages.

First of all, the heating obtained by the electromagnetic waves, even efficiently acting on the adipose tissue, causing the dissolving of the fats, does not penetrate in depth and does not interest the muscular tissue, that consequently is not damaged.

Advantageously, the cryogen cell, cooling the electrode placed on the body portion to be subjected to the treatment, protects the epidermis with respect to possible bums.

The combined use of the electromagnetic waves, to cause the heating, and of the cryogen cell, to cool the skin, allows to only intervene on the adipose tissue, without interesting the epidermis and the muscular tissue.

Advantageously, the presence of a movable and easily handy containing body allows to carry out the aesthetic treatment on any body zone, in an easy and clean way.

The so realised solution can be subjected to many variations and changes, all the modifications being included in the scope of the invention.

Furthermore, all the details can be replaced by technically equivalent elements, and the dimensions can be different, according to the needings.

Particularly, according to a different embodiment, radiofrequency generator can be replaced by another means suitable to heat the first electrode, or to induce a hating the subcutaneous adipose zone.

Said suitable means can be comprised, for example, by a microwave generator, by a laser generator, or by a high intensity ultrasound generator.

According to a further embodiment, the cryogen cell can be comprised by a cooling fluid (gas or liquid), cooling said first electrode 3, in place of said Peltier type electrical circuit.

According to a further embodiment, more than one active electrodes 3 can be provided, and a single passive electrode connected to the same, or more than one active and passive electrodes, all connected to the same generator.

1. Apparatus for lipolysis for aesthetic treatment, characterised in that it comprises:
at least a first electrode (3), or active electrode, that can be placed on a zone of the body to be subjected to the aesthetic treatment;
at least a second electrode (4), or passive electrode, bigger than the first electrode, that can be placed on a zone of the body contiguous, or neutral zone, to the treatment zone;
at least a generator (2) to heat the first electrode (3) to cause the heating of a subcutaneous adipose zone of the body to be subjected to aesthetic treatment; means (5, 6) for coupling the first electrode (3) and the second electrode (4) with the generator (2); thermo-adjustment means, operatively active in correspondence of the first electrode (3).

2. Apparatus according to claim 1, characterised in that it comprises at least a containing body (7) providing at least a handy portion (7a), said containing body (7) being movable and provided with at least a first end.

3. Apparatus according to claim 1, characterised in that the first electrode (3) is placed in correspondence of the first end of the containing body (7).

4. Apparatus according to claim 2, characterised in that said thermo-adjustment means are fixed to the containing body (7).

5. Apparatus according to claim 4, characterised in that said thermo-adjustment means comprises:
at least a cryogen cell (8) placed in correspondence of the first end of the containing body (7), said cryogen cell (8) being connected to the first electrode (3) and operatively active on the same;
a Peltier type circuit, housed within the containing body ( ) and operatively active on the cryogen cell (8).

6. Apparatus according to claim 5, characterised in that it further comprises means for cooling the electrical circuit.

7. Apparatus according to claim 6, characterised in that said means for cooling the electrical circuit comprises:
at least a duct (12) defining a closed circuit and containing cooling fluid, said duct (12) having at least a portion operatively active on the electrical circuit, to cool the same;
at least a pressure source (13) slaved to the closed circuit, to make the cooling fluid circulating within the same;
at least a heat exchanger (14) slaved to the closed circuit and serially placed with respect to said pressure source (13), to cool the cooling fluid.

8. Apparatus according to claim 1, wherein the generator (2) is a radiofrequency generator.

9. Apparatus according to claim 1, wherein the generator (2) is a microwave generator.

10. Apparatus according to claim 1, wherein the generator (2) is a laser generator.

11. Apparatus according to claim 1, wherein the generator (2) is a high intensity ultrasound generator.

12. Apparatus according to claim 1, wherein the cryogen cell (8) comprises a cooling fluid.

13. Method for lipolysis exclusively for aesthetic and not medical treatment, characterised in that it comprises the steps of:
applying a first electrode on a zone of the body to be subjected to the aesthetic treatment;
applying a second electrode, bigger than the first electrode, on a contiguous zone of the body, which is neutral with respect to the treatment;
cooling the first electrode and maintaining said cooling all along the aesthetic treatment;
heating the first electrode or heat induction into the subcutaneous adipose tissue of the body to be subjected to aesthetic treatment, after a pre-established period during which the above cooling were already active.

14. Method according to claim 14, wherein the treatment and the cooling last about 40 seconds.

15. Method according to claim 13, wherein the heating lasts about 20 seconds.
